(19) United States
(12) Patent Application Publication
(10) Pub. No.: US 2012/0046665 A1
(43) Pub. Date: Feb. 23, 2012

(54) ELECTRIC SCREWDRIVER FOR SPINAL FIXATION

(76) Inventor: Young Woo Kim, Seoul (KR)

(21) Appl. No.: 13/289,783

(22) Filed: Nov. 4, 2011

Related U.S. Application Data
(63) Continuation of application No. PCT/KR2010/001252, filed on Feb. 26, 2010.

(30) Foreign Application Priority Data
May 4, 2009 (KR) 10-2009-0038959

(51) Int. Cl. A61B 17/56

(52) U.S. Cl. 606/104

(57) ABSTRACT

An electric screwdriver that inserts a screw into the spine of a human and includes: a joint rod to which the screw and a motor is coupled, a power transmitting unit for transmitting the force of the motor to the joint rod, an electrical energy supply unit for supplying electrical energy to the motor, and a case, one end of which has a first holding portion to be held by one hand of a surgeon; a second holding portion coupled to an outer circumferential surface of the case such that the second holding portion is rotatable relative to the case and can be held by the other hand of the surgeon; and an automatic/manual operating unit which enables the second holding portion to be fixed when operating the motor, and rotatable when the motor stops operating and the surgeon manually inserts the screw.

### CROSS-REFERENCE TO RELATED APPLICATIONS

The present application is a continuation of pending International patent application PCT/KR2010/001252 filed on Feb. 26, 2010 which designates the United States and claims priority from Korean patent application 10-2009-0038959 filed on May 4, 2009, the content of which is incorporated herein by reference.

### FIELD OF THE INVENTION

The present invention relates to an electric screwdriver for spinal fixation, and more particularly, to an electric screwdriver for spinal fixation with which a surgeon may easily insert a screw into a spine.

### BACKGROUND OF THE INVENTION

Pedicle screw fixation is conducted in a spinal operation to fix the spine. In order to insert a certain screw into a spine fracture, the screw is inserted into spinal fractures above and below a spinal joint to be operated and then is firmly fixed.

In detail, a conventional electric screwdriver for spinal fixation includes a main body to which a joint rod for coupling a screw thereto is connected, and a first holding portion is formed at an end of the main body, which may be held by the hand of a surgeon; also, a second holding portion which has a cylindrical shape and is rotatably coupled to the main body is formed on an outer circumferential surface of the main body.

The conventional electric screwdriver for spinal fixation is designed to be supported while holding the second holding portion by the hand, and the first holding portion is rotated while holding the same such that a joint rod connected to the first holding portion is used to insert the screw into the spine and fix the screw.

However, the conventional electric screwdriver for spinal fixation has the following problems.

First, the surgeon has to manually rotate the screwdriver individually from the moment when a screw first contacts a spinal fracture until the screw is finally inserted into the spinal fracture to be fixed. Thus, it takes the surgeon much effort and also much time, accordingly.

Moreover, if the procedure is conducted on a patient who has bony tissues with a high bone density, into which a screw is inserted, the above disadvantages are intensified. In addition, if a screw is rotated manually, the screw might not be uniformly inserted at a predetermined desired angle and in a predetermined desired direction.

### SUMMARY OF THE INVENTION

The present invention provides an electric screwdriver for spinal fixation which enables an electrical insertion method for facilitating inserting a screw which is performed manually according to the conventional art, and whereby a screw may be inserted in a predetermined desired direction and at a predetermined desired angle, and a screw may also be manually inserted if required.

According to an aspect of the present invention, there is provided an electric screwdriver for spinal fixation which inserts a screw into the spine of a human body and comprises: a joint rod to one end of which the screw is coupled and the other end of which is coupled to a motor; a main body including a motor that is rotatable in the forward and reverse directions, a power transmitting unit for transmitting a rotating force of the motor to the joint rod, an electrical energy supply unit for supplying electrical energy to the motor, and a case, one end of which has a first holding portion to be held by one hand of a surgeon and which accommodates the motor, the power transmitting unit, and the electrical energy supply unit; a second holding portion coupled to an outer surface of the case such that the second holding portion is rotatable relative to the case and can be held by the other hand of the surgeon; and an automatic/manual operating unit which enables the second holding portion to be fixed on the case when the screw is inserted by operating the motor, and enables the second holding portion to be rotatable relative to the case when the motor stops operating and the surgeon manually inserts the screw.

The automatic/manual operating unit may comprise: a receiving portion formed on the second holding portion; and an engaging portion that is movable between a first position where the engaging portion is engaged with the receiving portion and a second position where the engagement is released.

The receiving portion may comprise a plurality of grooves formed in an inner surface of the second holding portion at predetermined intervals in a circumferential direction, and the engaging portion may comprise a protrusion protruding from an outer surface of a sliding part that is formed inside the case and is cylindrical and movable in a predetermined direction inside the case, wherein the protrusion has a cross-section corresponding to the grooves.

A power button that is inserted into the case when pressed to thereby supply power to the motor may be included in the electric energy supply unit, and a button press preventing portion that prevents the power button from being pressed when moved to the second position may be formed on the engaging portion.

The motor may operate at a low speed and a high torque.

An electric screwdriver for spinal fixation according to embodiments of the present invention enables an electric operation in which a screw may be automatically inserted as opposed to manually according to the conventional art. Thus, a surgeon does not have to manually rotate a screw individually to insert the same into a spine, which saves effort of the surgeon and minimizes an operation time.

In addition, as a screw is inserted electrically, the operation may be performed in a predetermined desired direction and at a predetermined desired angle, accurately and without shaking.

In addition, in the electric screwdriver for spinal fixation according to the embodiments of the present invention, in order that an operation may be performed manually according to operation conditions, an automatic/manual operating unit is additionally included so that a manipulation condition may be conveniently changed in case a motor is out of order, a battery runs out of power, or a screw has to be manually inserted precisely. Thus, the operation becomes easier to perform.

In addition, the electric screwdriver for spinal fixation according to the embodiments of the present invention includes a button press preventing portion that prevents a malfunction caused if a power button is pressed in a manual operation, thereby enabling a stable procedure.

Furthermore, the electric screwdriver for spinal fixation according to the embodiments of the present invention includes a motor that operates at a low speed and a high torque. Thus, damages to the spine of a patient due to a high rotational speed may be minimized.

### BRIEF DESCRIPTION OF THE DRAWINGS

FIG. 1 is a perspective view illustrating an electrical screwdriver for spinal fixation, according to an embodiment of the present invention;

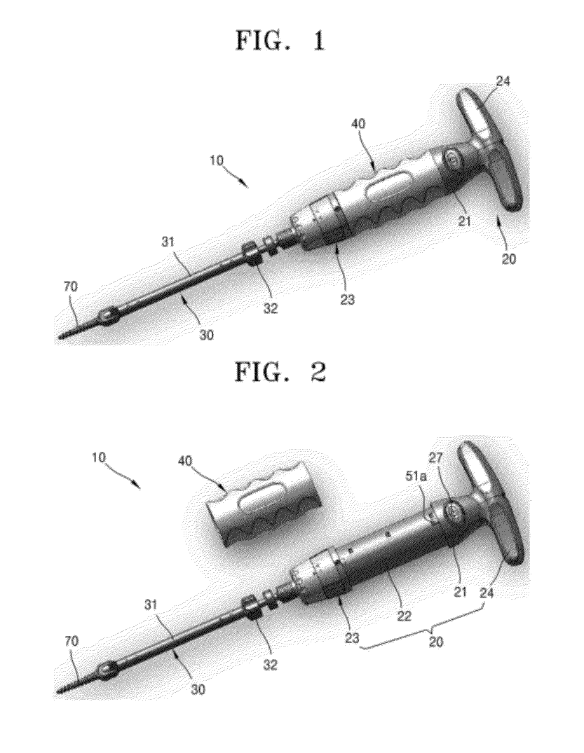

FIGS. 2 and 3 are perspective views of the disassembled views of the electrical screwdriver for spinal fixation of FIG. 1, according to an embodiment of the present invention;

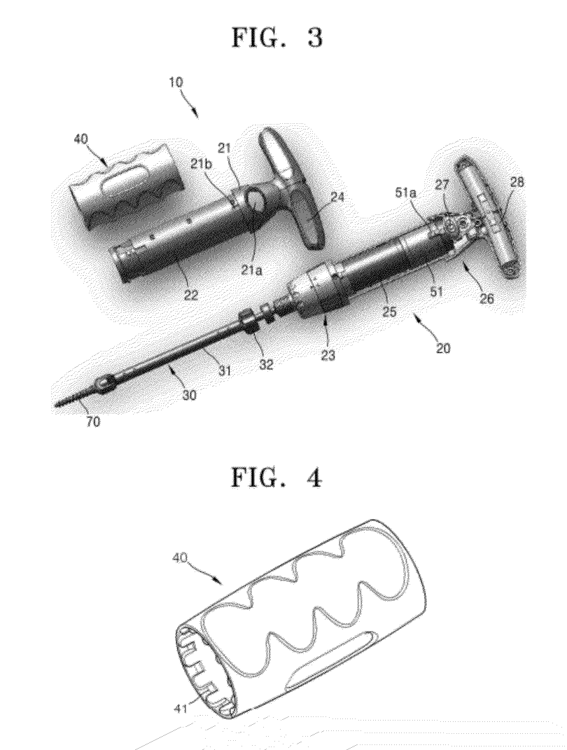

FIG. 4 is a perspective view illustrating a first holding portion among components illustrated in FIG. 1 according to an embodiment of the present invention;

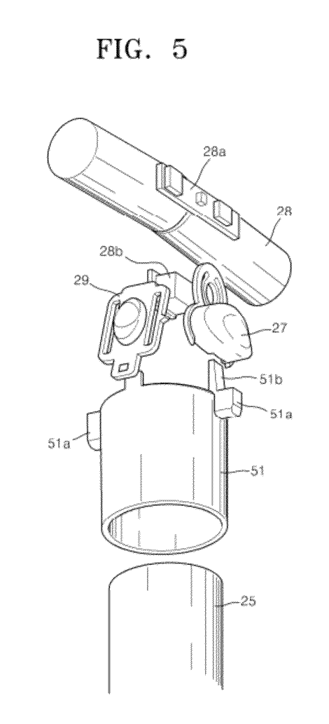

FIGS. 5 and 6 illustrate a main configuration of an automatic/manual operating unit of the electrical screwdriver for spinal fixation illustrated in FIG. 1 and an operation of the automatic/manual operating unit, according to an embodiment of the present invention; and

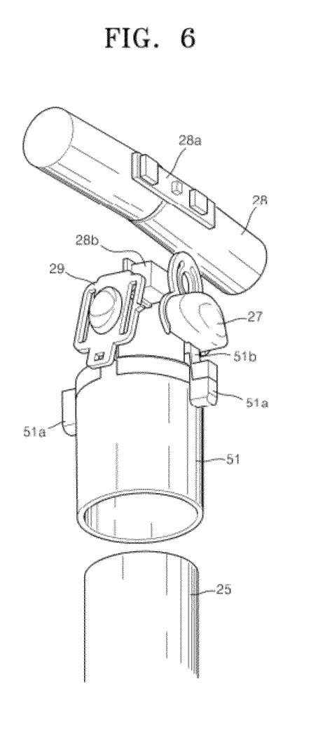

FIGS. 7 and 8 are cross-sectional views illustrating main components of a portion of the electrical screwdriver for spinal fixation illustrated in FIG. 1.

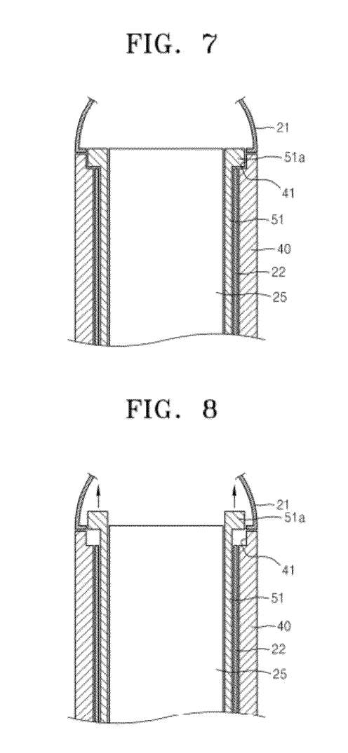

< Explanation of Reference Numerals >

| | | | |
|---|---|---|---|
| 10 | electrical screwdriver for | 20 | main body |
| | spinal fixation | | |
| 21 | first portion | 21a | first hole |
| 21b | second hole | 22 | bonding portion |
| 23 | second portion | 24 | first holding portion |
| 25 | motor | 26 | electrical energy supply unit |
| 27 | power button | 28 | battery |
| 29 | manipulation button | 30 | joint rod |
| 31 | axis portion | 32 | rotating protrusion |
| 40 | second holding portion | 41 | receiving portion |
| 51 | sliding part | 51a | engaging portion |
| 51b | button press preventing | 70 | screw |
| | portion | | |

### DETAILED DESCRIPTION OF THE INVENTION

A manual screwdriver used in pedicle screw fixation includes a T-shaped handle and a straight coupling portion for a screw, formed at an end portion of the screwdriver, wherein a small, low-speed and high torque electric motor is mounted around a portion of the T-shaped handle that is held by the hand to operate the screwdriver.

An electrical screwdriver for spinal fixation according to an embodiment of the present invention is designed to have the same form as a T-shaped driver used in pedicle screw fixation, and a small-sized straight-type electric motor of a low speed and a high torque is mounted around a handle so that the screwdriver may be operated by a button or a screw may be inserted manually.

When conducting multi-joint pedicle screw fixation, a screw is automatically inserted, and thus the screw may be inserted accurately, quickly, and easily, at a predetermined angle compared to when inserting a screw manually. Accordingly, an electric screwdriver for spinal fixation may be useful in spinal operations.

What is claimed is:

1. An electric screwdriver for spinal fixation which inserts a screw into the spine of a human body and comprises:
a joint rod to one end of which the screw is coupled and the other end of which is coupled to a motor;
a main body including a motor that is rotatable in the forward and reverse directions, a power transmitting unit for transmitting a rotating force of the motor to the joint rod, an electrical energy supply unit for supplying electrical energy to the motor, and a case, one end of which has a first holding portion to be held by one hand of a surgeon and which accommodates the motor, the power transmitting unit, and the electrical energy supply unit;
a second holding portion coupled to an outer circumferential surface of the case such that the second holding portion is rotatable relative to the case and can be held by the other hand of the surgeon; and
an automatic/manual operating unit which enables the second holding portion to be fixed on the case when the screw is inserted by operating the motor, and enables the second holding portion to be rotatable relative to the case when the motor stops operating and the surgeon manually inserts the screw.

2. The electric screwdriver for spinal fixation of claim 1, wherein the automatic/manual operating unit comprises:
a receiving portion formed on the second holding portion; and
an engaging portion that is movable between a first position where the engaging portion is engaged with the receiving portion and a second position where the engagement is released.

3. The electric screwdriver for spinal fixation of claim 2, wherein the receiving portion comprises a plurality of grooves formed in an inner surface of the second holding portion at predetermined intervals in a circumferential direction, and
the engaging portion comprises a protrusion protruding from an outer surface of a sliding part that is formed inside the case and is cylindrical and movable in a predetermined direction inside the case, wherein the protrusion has a cross-section corresponding to the grooves.

4. The electric screwdriver for spinal fixation of claim 3, wherein a power button that is inserted into the case when pressed to thereby supply power to the motor is included in the electric energy supply unit, and a button press preventing portion that prevents the power button from being pressed when moved to the second position is formed on the engaging portion.

5. The electric screwdriver for spinal fixation of claim 1, wherein the motor operates at a low speed and a high torque.
